(19) United States
(12) Patent Application Publication
(10) Pub. No.: US 2011/0152580 A1
(43) Pub. Date: Jun. 23, 2011

(54) PROCESS AND APPARATUS FOR VAPOR PHASE PURIFICATION DURING HYDROCHLORINATION OF MULTI-HYDROXYLATED ALIPHATIC HYDROCARBON COMPOUNDS

(76) Inventors: Bruce D. Hook, Lake Jackson, TX (US); Anna Forlin, Vigonza (IT); Andrei S. Merenov, Lake Jackson, TX (US); Danil Tirtowidjolo, Lake Jackson, TX (US); Anil J. Mehta, Lake Jackson, TX (US); Jan W. Verwijs, Hoek (NL); Aaltje Verwijs-van den Brink, Hoek (NL); Lena Verwijs, Hoek (NL); Hendrika Gerrita Verwijs, Hoek (NL); Wilma Hensen, Terneuzen (NL)

(21) Appl. No.: 12/595,488

(22) PCT Filed: Apr. 11, 2008
(86) PCT No.: PCT/US08/59980

§ 371 Date: May 7, 2010

Related U.S. Application Data
(60) Provisional application No. 60/923,019, filed on Apr. 12, 2007.

(51) Int. Cl. C07C 31/34; B01J 8/00

(52) U.S. Cl. 568/841; 422/187

(57) ABSTRACT

A process for converting multihydroxylated-aliphatic hydrocarbon compound(s) and/or ester(s) thereof to chlorohydrins and/or esters thereof is disclosed in which one or more of multihydroxylated-aliphatic hydrocarbon compound(s) and/or ester(s) thereof and/or monochlorohydrin(s) and/or ester(s) thereof with at least one chlorinating feed stream comprising at least one chlorinating agent and at least one impurity having a boiling point below the boiling point of the chlorohydrin product having the lowest boiling under hydrochlorination conditions, optionally in the presence of water, one or more catalyst(s), and/or one or more heavy byproduct(s) in a reaction vessel under hydrochlorination conditions, wherein the liquid-phase reaction mixture is maintained at a temperature below the boiling point of the chlorohydrin product having the lowest boiling point under hydrochlorination conditions and greater than the boiling point(s) of the at least one impurity and a vapor phase vent stream comprising the at least one impurity is removed from the liquid phase reaction mixture. An apparatus suitable for carrying out the disclosed process is illustrated in FIG. 1 of the drawings. The process and apparatus improve conversion rates and/or provide for recovery of chlorinating agent for lower operating costs.

### BACKGROUND OF THE INVENTION

The present invention relates to processes and apparatus for converting multihydroxylated-aliphatic hydrocarbon compound(s) and/or ester(s) thereof to chlorohydrins and/or esters thereof.

Dichlorohydrins are useful for making epoxides such as epichlorohydrins. Epichlorohydrin is a widely used precursor to epoxy resins. Epichlorohydrin is a monomer which is commonly used for the alkylation of para-bisphenol A. The resultant diepoxide, either as a free monomer or oligomeric diepoxide, may be advanced to high molecular weight resins which are used for example in electrical laminates, can coatings, automotive topcoats and clearcoats.

Glycerin is considered to be a low-cost, renewable feedstock that is a co-product of the biodiesel process for making fuel. It is known that other renewable feedstocks such as fructose, glucose and sorbitol can be hydrogenolized to produce mixtures of vicinal diols and triols, such as glycerin, ethylene glycol, 1,2-propylene glycol, 1,3-propylene glycol and the like. With abundant and low cost glycerin or mixed glycols, economically attractive processes for making dichlorohydrins from such feedstocks are desired.

A process is known for the conversion of glycerol (also referred to herein as “glycerin”) to mixtures of dichloropropanols, compounds I and II, as shown in Scheme 1 below. The reaction is carried out in the presence of anhydrous hydrogen chloride and an acetic acid (HOAc) catalyst with water removal. Compounds I and II can then be converted to epichlorohydrin via treatment with caustic or lime.

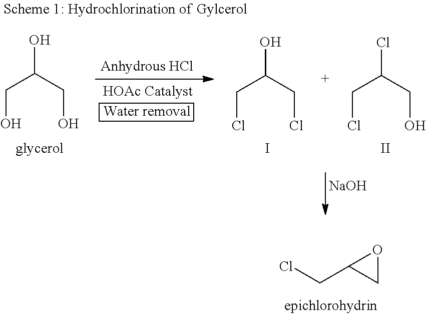

Various processes using the above chemistry in Scheme 1 have been reported in the prior art. For example, epichlorohydrin can be prepared by reacting a dichloropropanol such as 2,3-dichloro-1-propanol or 1,3-dichloro-2-propanol with base. Dichloropropanol, in turn, can be prepared at atmospheric pressure from glycerol, anhydrous hydrochloric acid, and an acid catalyst. A large excess of hydrogen chloride (HCl) was recommended to promote the azeotropic removal of water that is formed during the course of the reaction.

WO 2006/020234 A1 describes a process for conversion of a glycerol or an ester or a mixture thereof to a chlorohydrin, comprising the step of contacting a multihydroxylated-aliphatic hydrocarbon compound, an ester of a multihydroxylated-aliphatic hydrocarbon, or a mixture thereof with a source of a superatmospheric partial pressure of hydrogen chloride to produce chlorohydrins, esters of chlorohydrins, or mixtures thereof in the presence of an organic acid catalyst. Azeotropic removal of water via a large excess of hydrogen chloride is not required to obtain high chlorohydrins yield.

Likewise, WO2005021476(A1) describes the conversion of glycerin to dichloropropanol (dichlorohydrin) using gaseous hydrogen chloride with continuous removal of water at atmospheric pressure. WO2005021476(A1), however, does not describe the impact of impurities in the gaseous HCl on the process disclosed or the amount of HCl leaving the reactor with the impurities.

EP 1 752 435A1 discloses another process for producing a chlorohydrin by reaction between a multihydroxylated aliphatic hydrocarbon and/or an ester thereof and a chlorinating agent in which a vapor phase effluent stream is removed from a reactor for further processing to recover dichlorohydrin from the vapor phase. EP 1 752 435A1 discloses a process wherein a water/HCl/DCH mixture leaves as a vapor phase stream from a reactor and then rectifying that stream in a reactifier to return the HCl and DCH back to the reactor. The water is withdrawn as a condensate, but there is no mention in EP 1 752 435A1 of the impact of non-condensible gas impurities on the performance of that rectifier or the sizing impact of the non-condensibles.

CN 101007751A describes another process for producing a dichlorohydrin from glycerin. CN 101007751A describes the use of inert impurities being added to the HCl in order to improve the efficiency of stripping water from a reaction mixture in the reactor disclosed. CN 101007751A, however, does not address the increased loss of HCl from the reaction mixture resulting from this process arrangement, or the increased loss of HCl in the tailgas due to the high level of inerts passing through the condenser.

Opportunities remain to further improve the HCl yield in the processes of disclosed in the prior art.

### SUMMARY OF THE INVENTION

One aspect of the present invention is a process for making chlorohydrin(s) comprising contacting a liquid-phase reaction mixture comprising one or more of multihydroxylated-aliphatic hydrocarbon compound(s) and/or ester(s) thereof and/or monochlorohydrin(s) and/or ester(s) thereof with at least one chlorinating feed stream comprising at least one chlorinating agent and at least one impurity having a boiling point below the boiling point of the chlorohydrin product having the lowest boiling under hydrochlorination conditions, optionally in the presence of water, one or more catalyst(s), and/or one or more heavy byproduct(s) in a reaction vessel under hydrochlorination conditions, wherein
(a) the liquid-phase reaction mixture is maintained at a temperature below the boiling point of the chlorohydrin product having the lowest boiling point under hydrochlorination conditions and greater than the boiling point(s) of the at least one impurity and
(b) a vapor phase vent stream comprising the at least one impurity is removed from the liquid phase reaction mixture.

Another aspect of the present invention is an apparatus for making chlorohydrin(s) comprising:
(1) at least one reactor suitable for introducing a chlorinating feed stream comprising at least one chlorinating agent into a liquid reaction mixture comprising at least multihydroxylated-aliphatic hydrocarbon compound(s) and/or ester(s) thereof and/or monochlorohydrin(s) and/or ester(s) thereof and
(2) at least one vapor-liquid contacting device for recovering a chlorinating agent for recycle to at least one reactor,
wherein
at least one reactor (1) has at least one vent for removing impurities from a vapor phase within the reactor (1), the at least one contacting device (2) is connected to at least one reactor for conducting a stream comprising a fluid scrubbing agent combined and/or reacted with chlorinating agent to the at least one reactor, and
at least one vent is connected to the at least one contacting device (2) for conducting a vapor phase vent stream from the at least one reactor (1) to the at least one contacting device (2) for contacting a vapor phase vent stream with a scrubbing agent for removing chlorinating agent from a vapor phase vent stream; and/or
at least one reactor (1) is connected to the at least one contacting device (2) for conducting a chlorinated fluid scrubbing agent effluent from the at least one contacting device (2) to the at least one reactor (1) for introducing chlorinated fluid scrubbing agent effluent into a reaction mixture.

### BRIEF DESCRIPTION OF THE DRAWINGS

FIG. 1 is a schematic diagram illustrating the process and apparatus of the present invention.

FIG. 2 is a process block diagram illustrating a first embodiment of the process of the present invention.

FIG. 3 is a process block diagram illustrating a second embodiment of the process of the present invention.

FIG. 4 is a process block diagram illustrating a third embodiment of the process of the present invention.

FIG. 5 is a schematic diagram illustrating one preferred apparatus embodiment of the present invention.

FIG. 6 is a graphic representation of vent/feed flow ratio versus mass fraction impurity in the reactor headspace and vent at different feed impurity concentrations.

### DETAILED DESCRIPTION OF THE INVENTION

### Definitions

As used herein, the term “multihydroxylated-aliphatic hydrocarbon compound” (abbreviated hereafter as “MAHC”) refers to a compound that contains at least two hydroxyl groups covalently bonded to two separate vicinal carbon atoms and no ether linking groups. They contain at least two sp3 hybridized carbons each bearing an OH group. The MAHCs include any vicinal-diol (1,2-diol) or triol (1,2,3-triol) containing hydrocarbon including higher orders of contiguous or vicinal repeat units. The definition of MAHC also includes for example one or more 1,3- 1,4-, 1,5- and 1,6-diol functional groups as well. Geminal-diols, for example, are precluded from this class of MAHCs.

The MAHCs contain at least 2, preferably at least 3, up to about 60, preferably up to 20, more preferably up to 10, even more preferably up to 4, and yet more preferably up to 3, carbon atoms and can contain, in addition to aliphatic hydrocarbon, aromatic moieties or heteroatoms including for example halide, sulfur, phosphorus, nitrogen, oxygen, silicon, and boron heteroatoms; and mixtures thereof. The MAHCs may also be a polymer such as polyvinyl alcohol.

The multihydroxylated aliphatic hydrocarbon, the ester of a multihydroxylated aliphatic hydrocarbon, or the mixture thereof, according to the present invention may be a crude multihydroxylated aliphatic hydrocarbon, the ester of a crude multihydroxylated aliphatic hydrocarbon, or the mixture thereof; and may be obtained from renewable raw materials or biomass.

A “crude” multihydroxylated-aliphatic hydrocarbon product is a multihydroxylated-aliphatic hydrocarbon which has not been submitted to any treatment after its manufacture.

A “purified” multihydroxylated-aliphatic hydrocarbon product is a multihydroxylated-aliphatic hydrocarbon which has been submitted to at least one treatment after its manufacture.

“Renewable raw materials,” herein means materials designated as originating from the treatment of renewable natural resources. Among such materials, “natural” ethylene glycol, “natural” propylene glycol, and “natural” glycerol are preferred. Ethylene glycol, propylene glycol and “natural” glycerol are, for example, obtained via the conversion of sugars via known and unknown methods. As described in “Organic Chemistry, 3rd Ed. (Morrison & Boyd, Allyn & Bacon Publishers, 1973, pages 1070-1128)”, incorporated herein by reference, these sugars may come from, for example, sucrose sourced from crops such as cane or beets, amylose, glucose or maltose sourced from starch, or cellobiose sourced from cellulose. These sugars can also be obtained from biomass, as described in “Industrial Bioproducts; Today and Tomorrow, Energetics, Incorporated for the U.S. Department of Energy, Office of Energy Efficiency and Renewable Energy, Office of the Biomass Program, July 2003, pages 49, 52 to 56”, incorporated herein by reference.

The terms “glycerin”, “glycerol” and “glycerine”, and esters thereof, may be used as synonyms for the compound 1,2,3-trihydroxypropane, and esters thereof.

As used herein, the term “chlorohydrin” means a compound containing at least one hydroxyl group and at least one chlorine atom covalently bonded to two separate vicinal aliphatic carbon atoms and no ether linking groups. Chlorohydrins are obtainable by replacing one or more hydroxyl groups of MAHCs with covalently bonded chlorine atoms via hydrochlorination. The chlorohydrins contain at least 2, and preferably at least 3, up to about 60, preferably up to 20, more preferably up to 10, even more preferably up to 4, and yet more preferably up to 3, carbon atoms and, in addition to aliphatic hydrocarbon, can contain aromatic moieties or heteroatoms including for example halide, sulfur, phosphorus, nitrogen, oxygen, silicon, and boron heteroatoms, and mixtures thereof. A chlorohydrin that contains at least two hydroxyl groups is also a MAHC.

As used herein, the term “monochlorohydrin” means chlorohydrin having one chlorine atom and at least two hydroxyl groups, wherein the chlorine atom and at least one hydroxyl group are covalently bonded to two separate vicinal aliphatic carbon atoms (referred to hereafter by the abbreviation “MCH”). MCH produced by hydrochlorination of glycerin or glycerin esters includes, for example, 3-chloro-1,2-propanediol and 2-chloro-1,3-propanediol.

As used herein, the term “dichlorohydrin” means chlorohydrin having two chlorine atoms and at least one hydroxyl group, wherein at least one chlorine atom and at least one hydroxyl group are covalently bonded to two separate vicinal aliphatic carbon atoms (referred to hereafter by the abbreviation “DCH”). Dichlorohydrins produced by hydrochlorination of glycerin or glycerin esters include 1,3-dichloro-2-propanol and 2,3-dichloro-1-propanol.

As used herein, the expression “scrubbing agent” refers to a substance capable of reversibly or irreversibly combining with chlorine atoms, such as by reacting with, absorbing, or adsorbing chlorine atoms.

As used herein, the expression “chlorinated scrubbing agent” refers to a scrubbing agent combined with chlorine atoms. The chlorine atoms may be reacted with, absorbed by, or adsorbed by the scrubbing agent and may be bound to the scrubbing agent via covalent bonds, ionic attraction, or Van der Waal's forces. When the chlorine atoms are irreversibly combined with, or covalently bonded to, the scrubbing agent, the chlorinated scrubbing agents are preferably chlorohydrin(s).

As used herein, the expression “under hydrochlorination conditions” means conditions capable of converting at least 1 wt. %, preferably at least 5 wt. %, more preferably at least 10 wt. % of MAHCs, MCHs, and esters of MAHCs and MCHs present in a mixture and/or feed stream into DCH(s) and/or ester(s) thereof.

The expression “heavy byproduct(s)” refer to oligomers of mixture (a) components, such as oligomers of MAHCs and/or esters thereof and oligomers of chlorohydrins and/or esters thereof, and derivatives of such oligomers, such as esters thereof, chlorinated oligomers, and/or chlorinated esters thereof, having a number average molecular weight equal to or greater than the number average molecular weight of the oligomer, such as chlorinated oligomers. The terms chlorohydrin(s), MCH(s) and DCH(s), and ester(s) thereof, are not intended to include heavy byproducts.

The term “epoxide” means a compound containing at least one oxygen bridge on a carbon-carbon bond. Generally, the carbon atoms of the carbon-carbon bond are contiguous and the compound can include other atoms than carbon and oxygen atoms, like hydrogen and halogens, for example. Preferred epoxides are ethylene oxide, propylene oxide, glycidol and epichlorohydrin.

As used herein, the expression, “liquid phase” refers to a continuous intermediate phase between gas phase and a solid phase that may optionally comprise a minor amount of gas and/or solid discrete phase(s). The liquid phase may comprise one or more immiscible liquid phases and may contain one or more dissolved solids, such as one or more acids, bases, or salts.

As used herein, the expression “vapor phase” refers to a continuous gaseous phase that may optionally comprise a minor amount of liquid and/or solid discrete phase(s) (e.g., aerosol). The vapor phase may be a single gas or a mixture, such as a mixture of two or more gases, two or more liquid discrete phases, and/or two or more solid discrete phases.

The expression “lower boiling fraction” refers to a fraction derived from the mixture provided in step (a) in which more than half the total quantity of components of the lower boiling fraction are components of the mixture, or derived from the mixture, that are more volatile under the conditions of the unit operation than the components of the higher boiling fraction in the same unit operation derived from the same mixture provided in step (a).

The expression “higher boiling fraction” refers to a fraction derived from the mixture provided in step (a) in which more than half the total quantity of components of the higher boiling fraction are components of the mixture, or derived from the mixture, that are less volatile than the components of the lower boiling fraction in the same unit operation derived from the same mixture provided in step (a).

As used herein, the expression “liquid-vapor contacting device” refers to devices that serve to provide the contacting and development of at least one interfacial surface between liquid and vapor in the device. Examples of liquid-vapor contacting devices include plate column, packed column, wetted-wall (falling film) column, spray chamber, heat exchanger or any combination thereof. Examples of devices comprising plate columns and packed columns include distillation columns, fractionation columns, and stripping columns.

As used herein, the term “condenser” means a non-adiabatic system for removing heat from a process fluid via a secondary fluid physically separated from the process fluid. The process fluid and the secondary fluid may each be a vapor, a liquid, or a combination of liquid and vapor. A condenser is generally associated with a section of a distillation or fractionation column. It may be a unit operation external to a distillation column or it may be a unit operation internal to a distillation column. The physical separation may be in the form of tubes and the condensation may be carried out on the inside or outside of the tubes. The condenser may take the form of cooling elements on the decks of distillation column fractionating trays or as cooling elements between distillation column packing beds.

As used herein, the expression “plug flow reactor” refers to a reactor or system of reactors, optionally including associated equipment such as, for example, heat exchangers, piping, disengaging vessels, etc., for which the flow pattern through the reactor exhibits a plug flow residence time characteristic.

As used herein, the expression “a plug flow residence time characteristic” refers to a residence time distribution of the flow elements such that most of the fluid elements in a vessel or a system of vessels have approximately the same residence time and there is an existence of a composition profile along the flow path such that concentration of the reactants in the fluid decreases from inlet of the system to the outlet of the system and concentration of products increases from inlet of the plug flow system to the outlet of the plug flow system.

A reactor with a plug flow residence time characteristic is a reactor, or system of reactors and/or vessels, for which a step change in a component concentration to the inlet to the reactor or system of reactors and/or vessels is observed at the outlet of the reactor at some multiple of the average residence time of the reactor or system of reactors and/or vessels where 5% of the observed change occurs after at least 0.05 times the average residence time and where at least 87% of the observed change occurs after 2 times the average residence time.

O. Levenspiel, “The Chemical Reactor Omnibook” (Oregon State University Press, 1993), p. 64.12, provides further information regarding plug flow residence time and average residence time in plug flow reactors.

### Hydrochlorination of MAHCs:

Hydrochlorination may be conducted according to any hydrochlorination process well-known in the art. For example, German Patent No. 197308 teaches a process for preparing a chlorohydrin by the catalytic hydrochlorination of glycerin by means of anhydrous hydrogen chloride. WO 2005/021476 discloses a continuous process for preparing the dichloropropanols by hydrochlorination of glycerin and/or monochloropropanediols with gaseous hydrogen chloride with catalysis of a carboxylic acid. WO 2006/020234 A1 describes a process for conversion of a glycerol or an ester or a mixture thereof to a chlorohydrin, comprising the step of contacting a MAHC, an ester of a MAHC, or a mixture thereof with a source of a superatmospheric partial pressure of hydrogen chloride to produce a chlorohydrin, an ester of a chlorohydrin, or a mixture thereof in the presence of an organic acid catalyst without substantially removing water. The above references are hereby incorporated herein by reference with respect to the above-described disclosures.

In an exemplifying hydrochlorination process, MAHC and a hydrochlorination catalyst are charged to the hydrochlorination reactor. Then a chlorinating agent such as hydrogen chloride is added to the reactor. The reactor pressure is adjusted to the desired pressure and the reactor contents are heated to the desired temperature for the desired length of time. After completion of the hydrochlorination reaction or while carrying out the hydrochlorination reaction, the reactor contents as a reaction effluent stream is discharged from the reactor and fed directly, or indirectly via another reactor or other intervening step, to a separation system comprising a DCH recovery system according to the present invention and optionally including other separation systems or equipment, such as a flash vessel and/or reboiler.

The hydrochlorination reaction above may be carried out in one or more hydrochlorination reactor vessels such as a single or multiple continuous stirred tank reactors (referred to hereafter by the abbreviation “CSTR”), single or multiple tubular reactor(s), cylindrical reactor(s), bubble column reactor(s), trayed distillation tower reactor(s), plug flow reactor(s) (referred to hereafter by the abbreviation “PFR(s)”), spray tower(s), venturi eductor(s), heat exchanger(s), falling film contactor(s) or combinations thereof. The hydrochlorination reactor can be, for example, one reactor or multiple reactors connected with each other in series or in parallel including, for example, one or more CSTRs, one or more tubular reactors, one or more PFRs, one or more bubble column reactors, and combinations thereof.

The equipment useful for conducting the hydrochlorination reaction may be any well-known equipment in the art and should be capable of containing the reaction mixture at the conditions of the hydrochlorination. Suitable equipment may be fabricated of materials which are resistant to corrosion by the process components, and may include for example, metals such as tantalum, suitable metallic alloys (particularly nickel-molybdenum alloys such as Hastalloy C©), or glass-lined equipment, for example.

In addition to DCH(s), one or more of the unreacted MAHC(s) and/or chlorination agent(s), reaction intermediates such as MCH(s), MCH ester(s), and/or DCH ester(s), catalyst(s), ester(s) of catalyst(s), water, and/or heavy byproduct(s) may present in mixture (a). A recycle process is preferred in which one or more of the unreacted MAHC(s), ester(s) of MAHC(s) and/or chlorination agent(s), reaction intermediates such as MCH(s), MCH ester(s), DCH ester(s), and other substances such as catalyst(s), ester(s) of catalyst(s), and water are preferably recycled to the process from a downstream unit operation for further hydrochlorination. Such recycle process(es) is preferably continuous. In this manner, raw material efficiencies are maximized and/or catalysts are reused.

When catalysts are reused in such a process scheme, it may be desirable to employ the catalysts in a higher concentration than they are employed in a single-pass process. This may result in faster reactions, or smaller process equipment, which results in lower capital costs for the equipment employed.

In a continuous recycle process, undesirable impurities and/or reaction byproducts may build up in the process. Thus, it is desirable to provide a means for removing such impurities from the process, such as via one or more vent outlets, for example, or by a separation step. Furthermore, a purged stream may be further treated to recover a useful portion of the purged stream.

The chlorinating agent that may optionally be present in the mixture treated according to the present invention is preferably hydrogen chloride or hydrogen chloride source, and may be a gas, a liquid or in a solution, or a mixture thereof. The hydrogen chloride is preferably introduced in the gaseous state and, when the hydrochlorination reaction mixture is in the liquid phase, at least some of the hydrogen chloride gas is preferably dissolved in the liquid reaction mixture. The hydrogen chloride may, however, be diluted in a solvent, such as an alcohol (for example methanol) or a chlorinated hydrocarbon, or in a carrier gas such as nitrogen, if desired.

It is preferred that hydrochlorination be carried out under superatmospheric pressure conditions. “Superatmospheric pressure” herein means that the hydrogen chloride (HCl) partial pressure is above atmospheric pressure, i.e. 15 psia (103 kPa) or greater. Generally, the hydrogen chloride partial pressure employed in the hydrochlorination process is at least about 15 psia (103 kPa) or greater. Preferably, the hydrogen chloride partial pressure employed in the hydrochlorination process is not less than about 25 psia (172 kPa), more preferably not less than about 35 psia (241 kPa), and most preferably not less than about 55 psia (379 kPa); and preferably not greater than about 1000 psia (6.9 MPa), more preferably not greater than about 600 psia (4.1 MPa), and most preferably not greater than about 150 psia (1.0 MPa).

The reaction of the superatmospheric pressure process of the present invention is advantageously rapid and may be carried out for a time period of less than about 12 hours, preferably less than about 5 hours, more preferably less than about 3 hours and most preferably less than about 2 hours. At longer reaction times, such as above about 12 hours, the process begins to form RCIs and other over-chlorinated by-products.

High per-pass yields and high selectivity can be achieved using the superatmospheric pressure process of the present invention. For example, a per-pass yield for the chlorohydrin based on the multihydroxylated-aliphatic hydrocarbon of greater than about 80%, preferably greater than about 85%, more preferably greater than about 90%, and most preferably greater than about 93% can be achieved by the present invention. For example, a high selectivity of greater than about 80%, preferably greater than about 85%, more preferably greater than about 90%, and most preferably greater than about 93% of chlorohydrins can be achieved by the process of the present invention. Of course, yields can be increased by recycling reaction intermediates.

Still another embodiment of the present invention relates to a batch, semi-batch, continuous or semi-continuous process for preparing a dichlorohydrin of glycerin comprising the step of contacting together at a superatmospheric partial pressure of HCl, for example in the range of from about 20 psia to about 1000 psia; and at a sufficient temperature, for example in the range of from about 25° C. to about 300° C.: (a) an ester of a multihydroxylated-aliphatic hydrocarbon, for example glycerin monoacetate; and (b) a hydrogen chloride source, for example hydrogen chloride; wherein the process is carried out without substantially removing water during the contacting or reaction step.

For the process of converting MAHCs to chlorohydrins, hydrogen chloride is required in a form that provides sufficient reactive activity for the hydrochlorination reaction to occur. Sources of hydrogen chloride include production of methylene-di(phenylisocyanate) (MDI), toluene diisocyanate (TDI), vinyl chloride (VCM), ethylene dichloride, perchloroethylene, chlorinated methanes, chlorinated propenes, chlorinated propanes aliphatic isocyanates, and other processes using phosgene for carbonylation, chlorinated ethanes, 1,3 dichloropropene and allyl chloride. These sources provide hydrogen chloride as a byproduct of their processes, typically as an anhydrous gas. Impurities in the gas provided may include but are not limited to nitrogen, carbon monoxide, carbon dioxide, phosgene, chlorinated benzenes, other chlorinated organic compounds (chloropropenes, allyl chloride, chloropropanes, chloromethanes, chlorinated ethanes, chlorobutanes, vinyl chloride, vinylidene chloride, monochloropropenes, perchloroethylene, trichloroethylene, chlorobutadiene, chlorobenzenes and their mixture), methanol, methane, ethane, ethylene, acetylene, propane, propylene, butanes, butylenes and other aliphatic and olefinic compounds. The processes from which the chlorinating agent may originate are often associated with the process. The heavy byproducts of the synthesis of the allyl chloride and epichlorohydrin are advantageously used as raw material sources in the chlorinolysis at high temperatures in order to produce valuable commercial materials. These installations may, however, have other sources of raw materials. An oxidation at a temperature greater than or equal to 800° C. is used to eliminate all the chlorinated or oxygenated organic waste.

The processes from which the chlorination agent may originate generate hydrogen chloride or aqueous solutions of hydrogen chloride as co-products. These acids are generally of mediocre quality containing traces of organic materials. They are advantageously used in the method for the production of the chlorohydrin mentioned above as is or after treatment. These acid streams likewise may contain inert compounds such as nitrogen (N₂), carbon monoxide (CO), carbon dioxide (CO₂), argon (Ar), oxygen (O₂), or other trace gases, as well as trace organic materials.

Surprisingly, these impurities are largely unaffected by the MAHC hydrochlorination reaction mechanism, are largely insoluble in the liquid phase and gather in the headspace of the reactor used for the chlorination, or in the gas phase of the reactor. Consequently, insufficient venting of the headspace, even for hydrogen chloride streams of high purity, can result in vapor phase partial pressures of hydrogen chloride that are fractions of the total pressure of the vapor phase. This has detrimental effect on the hydrogen chloride liquid phase concentration available for hydrochlorination reactions, and can significantly slow the rate of reaction.

Conversely, recognizing the partial pressure effect can result in requiring vessel designs with pressure ratings far in excess of the hydrogen chloride partial pressure desired to drive the reaction.

Venting of large amounts of the hydrogen chloride in order to reduce the impurity level in the headspace can result in significant economic loss due to the requirement to scrub the vent stream with caustic or other alkaline or alkaline earth metal source. Scrubbing the hydrogen chloride with caustic makes it unusable for hydrochlorination, in addition to requiring a costly source of neutralizing agent (caustic or other).

One aspect of the present invention is to reduce hydrogen chloride loss to the hydrochlorination process by recovering the hydrogen chloride lost by venting through reaction, absorbtion and/or adsorption into another process stream that can be returned to the reactor, while allowing the uncondensed, undissolved, inert or unreactive components in the hydrogen chloride feed to be discharged from the process with minimal loss of reactant.

In one aspect of this invention, the vapor stream leaving the hydrochlorination reactor is contacted with one or more MAHC(s), such as glycerin, which after the hydrogen chloride is combined with it, is then fed to the hydrochlorination reactor or a subsequent hydrochlorination reactor or a separate hydrochlorination reactor. The glycerin may be a purified glycerin, a crude glycerin, or a process stream containing glycerin.

In a second aspect of this invention, the vapor stream leaving the hydrochlorination reactor is contacted with a stream including, among others, one or more MAHC(s), such as glycerin, and optionally one or more catalyst(s), or mixture thereof, which after the hydrogen chloride is reacted, absorbed, or adsorbed into it, it is then fed to the hydrochlorination reactor that is the source of the vent stream or to another hydrochlorination reactor upstream or downstream from the hydrochlorination reactor that is the source of the vent stream. The glycerin may be a purified glycerin, a crude glycerin, or a process stream containing glycerin.

In a third aspect of this invention, the hydrochlorination process contains one or more hydrochlorination reactors, followed by a separation device that separates the chlorohydrin product from the underchlorinated or unreacted MAHC or glycerin, organic acid catalyst, or mixture thereof. A recycle stream containing the underchlorinated or unreacted MAHC or glycerin, organic acid catalyst, or mixture thereof, is used as the reactant, absorbent and/or adsorbent to react with, absorb and/or adsorb the hydrogen chloride and then feed it back to the reactor from which it was vented.

The temperatures useful in the practice of the process of the present invention are sufficient to give economical reaction rates, but not so high that starting material, product or catalyst stability becomes compromised. Furthermore, high temperatures increase the rate of undesirable uncatalyzed reactions, such as non-selective over-chlorination, and can result in increased rates of equipment corrosion. Useful temperatures in the present invention generally may be preferably at least about 25° C. more preferably at least about 60° C., even more preferably at least about 80° C. and most preferably at least about 100° C. It is preferable to keep the temperature below about 300° C., preferably below about 200° C., more preferably to about 160° C., even more preferably to about 150° C. and most preferably to about 120° C.

It is preferred to conduct the hydrochlorination step at a temperature sufficient for hydrochlorination that is also below the boiling point of the chlorohydrin(s) in the reaction mixture having the lowest boiling point for a given pressure condition during the hydrochlorination step in order to keep the chlorohydrin(s) produced and converted during hydrochlorination in the liquid phase of the reaction mixture for recovery in steps (b) and (c). The upper limit of this preferred temperature range may be adjusted by adjusting the pressure condition. A higher pressure during hydrochlorination may be selected to increase the boiling point temperature of the chlorohydrin(s) in the reaction mixture, so that the preferred temperature range for keeping DCH(s) in the liquid phase may be increased by increasing the pressure condition.

MAHCs fed to the reaction mixture may include for example 1,2-ethanediol; 1,2-propanediol; 1,3-propanediol; 3-chloro-1,2-propanediol; 2-chloro-1,3-propanediol; 1,4-butanediol; 1,5-pentanediol; cyclohexanediols; 1,2-butanediol; 1,2-cyclohexanedimethanol; 1,2,3-propanetriol (also known as, and used herein interchangeable as, “glycerin”, “glycerine”, or “glycerol”); and mixtures thereof. Preferably, the MAHCs in the effluents treated according to the present invention include for example 1,2-ethanediol; 1,2-propanediol; 1,3-propanediol; and 1,2,3-propanetriol; with 1,2,3-propanetriol being most preferred.

Examples of esters of MAHCs found in the effluent treated according the present invention include for example ethylene glycol monoacetate, propanediol monoacetates, glycerin monoacetates, glycerin monostearates, glycerin diacetates, and mixtures thereof. In one embodiment, such esters can be made from mixtures of MAHC with exhaustively esterified MAHC, for example mixtures of glycerol triacetate and glycerol.

One particularly suitable glycerol useful in the present invention may be obtained during the transformation of animal fats. Another particularly suitable glycerol, which may be used in the present invention, can be obtained during the production of oleochemicals or biodiesel. Still another suitable glycerol for use in the present invention can be obtained during the transformation of fats or oils—animal or vegetable—via trans-esterification in the presence of a heterogeneous catalyst, as described in FR 2752242, FR 2869612, and FR 2869613, each incorporated herein by reference. In such process it is advantageous to use a heterogeneous catalyst comprising mixed aluminum and zinc oxides, mixed zinc and titan oxides, mixed zinc, titan and aluminum oxides, and the mixed bismuth and aluminum oxides; and mixtures thereof. The heterogeneous catalyst may be operated in a fixed bed. This latter method, for example, can be a biodiesel production method.

Glycerol sourced from production of oleochemicals or biodiesel may be advantageously used in the present invention because of its lower cost when taken as crude glycerol, or when taken as un-neutralized crude glycerol. As described in “Process Economics Program Report 251, Biodiesel Production (October 2004), (R. G. Bray, SRI Consulting, pp. 7-10 to 7-14)”, incorporated herein by reference, the alkaline catalyzed transesterification of oils or fats with alcohols to alkyl esters and glycerol results in a two phase mixture of alkaline-containing glycerol and alkyl esters. The crude mixtures of multihydroxylated-aliphatic hydrocarbons of the present invention may be used in any desirable non-limiting concentration. In general, higher concentrations are preferred for economic reasons. Useful concentrations for the multihydroxylated-aliphatic hydrocarbons of the present invention may include, for example from about 0.01 mole % to about 99.99 mole %, preferably from about 1 mole % to about 99.5 mole %, more preferably from about 5 mole % to about 99 mole %, and most preferably from about 10 mole % to about 95 mole %.

In one embodiment of the process according to the present invention, the crude multihydroxylated aliphatic hydrocarbon product may generally comprise at least 40% by weight of the multihydroxylated-aliphatic hydrocarbon. Often, the crude product comprises at least 50% by weight of the multihydroxylated-aliphatic hydrocarbon. Preferably, it comprises at least 70% by weight of the multihydroxylated-aliphatic hydrocarbon. Often, the crude product comprises at most 99% by weight of the multihydroxylated-aliphatic hydrocarbon. Typically, it comprises at most 95% by weight of the multihydroxylated-aliphatic hydrocarbon.

In another embodiment of the present invention, the crude multihydroxylated-aliphatic hydrocarbon product comprises at most 89% by weight of the multihydroxylated-aliphatic hydrocarbon. In that embodiment, the crude multihydroxylated-aliphatic hydrocarbon product comprises at most 85% by weight of the multihydroxylated-aliphatic hydrocarbon. In that embodiment, the crude multihydroxylated-aliphatic hydrocarbon product comprises generally at least 10% by weight of water and often at least 14% by weight of water.

The MCHs generally correspond to the hydrochlorinated MAHCs in which one of a pair of hydroxyl groups covalently bonded to two separate vicinal carbon atoms is replaced by a covalently bonded chlorine atom. The ester(s) of MCH may be the result of hydrochlorination of MAHC ester(s) or reaction with an acid catalyst, for example.

The DCHs generally correspond to the hydrochlorinated MAHCs in which two hydroxyl groups covalently bonded to two separate carbon atoms, at least one of which is vicinal to a third carbon atom having a hydroxyl group, are each replaced by a covalently bonded chlorine atom. The ester(s) of DCH may be the result of hydrochlorination of MAHC ester(s), MCH ester(s) or reaction(s) with acid catalyst(s), for example.

In an embodiment of the present invention where MAHC(s) is/are the starting material fed to the process, as opposed to ester(s) of MAHC(s) or a mixture of MAHC(s) and ester(s) thereof as a starting material, it is generally preferred that the formation of chlorohydrin be promoted by the presence of one or more catalyst(s) and/or ester(s) thereof. Catalyst(s) and/or ester(s) thereof may also be present where ester(s) of MAHC(s), or a mixture of MAHC(s) and ester(s) thereof, is a starting material to further accelerate the hydrochlorination reaction.

Carboxylic acids, RCOOH, catalyze the hydrochlorination of MAHCs to chlorohydrins. The specific carboxylic acid catalyst chosen may be based upon a number of factors including for example, its efficacy as a catalyst, its cost, its stability to reaction conditions, and its physical properties. The particular process, and process scheme in which the catalyst is to be employed may also be a factor in selecting the particular catalyst. The “R” groups of the carboxylic acid may be independently chosen from hydrogen or hydrocarbyl groups, including alkyl, aryl, aralkyl, and alkaryl. The hydrocarbyl groups may be linear, branched or cyclic, and may be substituted or un-substituted. Permissible substituents include any functional group that does not detrimentally interfere with the performance of the catalyst, and may include heteroatoms. Non-limiting examples of permissible functional groups include chloride, bromide, iodide, hydroxyl, phenol, ether, amide, primary amine, secondary amine, tertiary amine, quaternary ammonium, sulfonate, sulfonic acid, phosphonate, and phosphonic acid.

The carboxylic acids useful as hydrochlorination catalysts may be monobasic such as acetic acid, formic acid, propionic acid, butyric acid, isobutyric acid, hexanoic acid, 4-methylvaleric acid, heptanoic acid, oleic acid, or stearic acid; or polybasic such as succinic acid, adipic acid, or terephthalic acid. Examples of aralkyl carboxylic acids include phenylacetic acid and 4-aminophenylacetic acid. Examples of substituted carboxylic acids include 4-aminobutyric acid, 4-dimethylaminobutyric acid, 6-aminocaproic acid, 6-hydroxyhexanoic acid, 6-chlorohexanoic acid, 6-aminohexanoic acid, 4-aminophenylacetic acid, 4-hydroxyphenylacetic acid, lactic acid, glycolic acid, 4-dimethylaminobutyric acid, and 4-trimethylammoniumbutyric acid. Additionally, materials that can be converted into carboxylic acids under reaction conditions, including for example carboxylic acid halides, such as acetyl chloride, 6-chlorohexanoyl chloride, 6-hydroxyhexanoyl chloride, 6-hydroxyhexanoic acid, and 4-trimethylammonium butyric acid chloride; carboxylic acid anhydrides such as acetic anhydride and maleic anhydride; carboxylic acid esters such as methyl acetate, methyl propionate, methyl pivalate, methyl butyrate, ethylene glycol monoacetate, ethylene glycol diacetate, propanediol monoacetates, propanediol diacetates, glycerin monoacetates, glycerin diacetates, glycerin triacetate, and glycerin esters of a carboxylic acid (including glycerin mono-, di-, and tri-esters); MAHC acetates such as glycerol 1,2-diacetate; carboxylic acid amides such as ε-caprolactam and γ-butyrolactam; and carboxylic acid lactones such as γ-butyrolactone, δ-valerolactone and ε-caprolactone may also be employed in the present invention. Zinc acetate is an example of a metal organic compound. Mixtures of the foregoing catalysts and catalyst precursors may also be used.

When a catalyst is used in the superatmospheric pressure process, the catalyst may be for example a carboxylic acid; an anhydride; an acid chloride; an ester; a lactone; a lactam; an amide; a metal organic compound such as sodium acetate; or a combination thereof. Any compound that is convertible to a carboxylic acid or a functionalized carboxylic acid under hydrochlorination reaction conditions may also be used. A preferred carboxylic acid for the superatmospheric pressure process is an acid with a functional group consisting of a halogen, an amine, an alcohol, an alkylated amine, a sulfhydryl, an aryl group or an alkyl group, or combinations thereof, wherein this moiety does not sterically hinder the carboxylic acid group.

Certain catalysts may also be advantageously employed at superatmospheric, atmospheric or sub-atmospheric pressure, and particularly in circumstances where water is continuously or periodically removed from the reaction mixture to drive conversion to desirably higher levels as may be the case when recovering DCH(s) according to the claimed invention. For example, the hydrochlorination of MAHC(s) reaction can be practiced by introducing hydrogen chloride gas into contact with a mixture of MAHC(s) and catalyst(s), such as by sparging the hydrogen chloride gas through a liquid phase reaction mixture. In such a process, the use of less volatile catalysts, such as 6-hydroxyhexanoic acid, 4-aminobutyric acid; dimethyl 4-aminobutyric acid; 6-chlorohexanoic acid; caprolactone; carboxylic acid amides such as ε-caprolactam and γ-butyrolactam; carboxylic acid lactones such as γ-butyrolactone, δ-valerolactone and ε-caprolactone; caprolactam; 4-hydroxyphenyl acetic acid; 6-amino-caproic acid; 4-aminophenylacetic acid; lactic acid; glycolic acid; 4-dimethylamino-butyric acid; 4-trimethylammoniumbutyric acid; and combination thereof; and the like may be preferred. It is most desirable to employ a catalyst, under these atmospheric or subatmospheric conditions, that is less volatile than the DCH(s) produced and recovered.

Preferred catalysts used in the present invention include carboxylic acids, esters of carboxylic acids, and combinations thereof, particularly esters and acids having a boiling point higher than that of the desired highest boiling DCH that is formed in the reaction mixture (i.e., the catalyst(s) is/are preferably less volatile than the DCH(s) in the mixture), so that the DCH(s) can be removed without removing the catalyst. Catalysts which meet this definition and are useful in the present invention include for example, polyacrylic acid, glycerin esters of carboxylic acids (including glycerin mono-, di-, and tri-esters), polyethylene grafted with acrylic acid, divinylbenzene/methacrylic acid copolymer, 6-chlorohexanoic acid, 4-chlorobutanoic acid, caprolactone, heptanoic acid, 4-hydroxyphenylacetic acid, 4-aminophenylacetic acid, 6-hydroxyhexanoic acid, 4-aminobutyric acid, 4-dimethylaminobutyric acid, 4-trimethyl-ammoniumbutyric acid chloride, stearic acid, 5-chlorovaleric acid, 6-hydroxyhexanoic acid, 4-aminophenylacetic acid, and mixtures thereof. Carboxylic acids that are sterically unencumbered around the carboxylic acid group are generally preferred.

Furthermore, the catalyst(s) is/are preferably miscible with the MAHC(s) employed. For this reason, the catalyst(s) may contain polar heteroatom substituents such as hydroxyl, amino or substituted amino, or halide groups, which render the catalyst miscible with the MAHC(s) in the reaction mixture, such as glycerol.

One embodiment of the catalyst that may be present is generally represented by Formula (a) shown below wherein the functional group “R′” includes a functional group comprising an amine, an alcohol, a halogen, a sulfhydryl, an ether; or an alkyl, an aryl or alkaryl group of from 1 to about 20 carbon atoms containing said functional group; or a combination thereof; and wherein the functional group “R” may include a hydrogen, an alkali, an alkali earth or a transition metal or a hydrocarbon functional group.

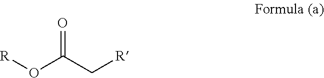

Where the catalyst is recycled and used repeatedly, such recycled catalysts may be present in an amount from about 0.1 mole %, preferably from about 1 mole %, more preferably from about 5 mole %, up to about 99.9 mole %, preferably up to 70 mol %, and more preferably up to 50 mole %, based on the amount in moles of MAHC present. Higher catalysts concentrations may be desirably employed to reduce the reaction time and minimize the size of process equipment.

In a preferred embodiment, the mixture (a) comprises water, such as the water produced as a byproduct of the hydrochlorination reaction, water present in the starting materials for the hydrochlorination reaction, and/or water recycled from a downstream unit operation. The mixture (a) may contain at least 1, more preferably at least 5, weight-percent water up to 90, more preferably up to 50, weight-percent water.

The above process may be carried out continuously or discontinuously. The above process is preferably carried out continuously (i.e., without interruption) for a time period of at least one hour.

Recovery of Chlorinating Agent from the Vent Stream

The feed flow rate of the chlorinating agent introduced into the reaction vessel is preferably more than 1 percent, more preferably at least four times, and even more preferably at least 10 times, greater than the flow rate of the chlorinating agent in the vapor phase vent stream.

In a preferred embodiment, the process further comprises:
(c) the vapor phase vent stream is contacted with a fluid scrubbing agent capable of adsorbing or reacting with the chlorinating agent(s) to remove chlorinating agent(s) from the vapor phase vent stream or
(c′) the chlorinating agent feed stream is contacted with a fluid scrubbing agent capable of adsorbing or reacting with the chlorinating agent(s) to remove chlorinating agent(s) from the chlorinating agent feed stream and optionally
(d) at least a fraction of the scrubbing agent is introduced into a hydrochlorination reaction after step (c) or (c′).

In one embodiment the scrubbing agent of step (c) comprises a solvent. Preferred solvents include alcohols, halogenated fluids, ethers, saturated hydrocarbons and mixtures thereof. Halogenated fluids may include DCHs and esters thereof.

In the same or alternative embodiment, the scrubbing agent is capable of reacting with chlorinating agent from the vapor phase vent stream to form chlorohydrin(s). Preferred scrubbing agents according to this embodiment include any of the MAHCs, MCHs, and/or esters of MAHCs and MCHs identified above as suitable for use in the reaction mixture for making chlorohydrins(s) and/or ester(s) thereof. The MAHC(s) and/or MCH(s) are preferably the same as those contained in the reaction mixture.

In one preferred embodiment, the reaction mixture is removed from the reaction vessel, at least some of the chlorinating agent(s) and dichlorohydrin(s) in the reaction mixture are removed from the reaction mixture in one or more unit operations, and the reaction mixture residue depleted of chlorinating agent(s) and dichlorohydrin(s) is used as the scrubbing agent.

The fluid scrubbing agent may further comprise catalysts or esters thereof. The catalysts are preferably present when the scrubbing agent comprises MAHC(s), MCH(s), or ester(s) of MAHC(s) or MCH(s), particularly when non-ester(s) of MAHC(s) and/or MCH(s) are present in the fluid scrubbing agent. The catalyst(s) and/or ester(s) thereof may be selected from those identified above a suitable for the reaction mixture. The catalyst(s) and/or ester(s) thereof in the fluid scrubbing agent is/are preferably the same as one or more catalyst(s) or ester(s) thereof present in the reaction mixture.

The scrubbing agent is preferably introduced as a feed stream flowing in counter-current flow direction relative to the vapor phase vent stream or chlorinating agent feed stream.

In one embodiment, the contacting is carried out via reactive distillation.

The reaction vessel may be a continuous stirred tank reactor, a tubular reactor, a cylindrical reactor, a bubble column reactor, a packed tower, a trayed tower, a spray tower, a venturi eductor, a heat exchanger, a falling film contactor or any combination thereof.

The reaction vessel is preferably the source of the vapor phase vent stream and at least a fraction of the chlorinated fluid scrubbing agent effluent from the liquid-vapor contacting device is preferably introduced into the reaction vessel that is the source of the vapor phase vent stream for the liquid-vapor contacting device according to step (c). The source of the vapor phase vent stream may be a continuous stirred tank reactor, a bubble column reactor, or a trayed distillation tower reactor, for example.

The source of the vapor phase vent stream may also, or alternatively, be a flash vessel downstream from the at least one reactor. The scrubbing agent may be introduced into at least one downstream reaction vessel connected to the reaction vessel that is the source of the vapor phase vent stream. The at least one downstream reaction vessel may comprise at least one reactor having a plug flow residence time characteristic.

The steps of the process are preferably carried out simultaneously and continuously for at least one hour.

The apparatus preferably comprises at least one separation vessel (3) for separating at least some of the chlorinating agent(s) and dichlorohydrin(s) from unreacted and partially reacted reactants connected to the at least one reactor (1) for conducting a liquid reaction mixture effluent stream from the at least one reactor (1) to the at least one separation vessel (3).

The at least one separation vessel (3) is preferably connected to the contacting device (2) for conducting a stream comprising at least a fraction of a liquid residue after separation of chlorinating agent(s) and dichlorohydrin(s) from the at least one separation vessel (3) to the at least one contacting device (2) for use as a fluid scrubbing agent for removing chlorinating agent from a vapor phase vent stream.

The contacting device (2) preferably comprises a scrubbing column, a falling film absorber, or any combination thereof.

The contacting device (2) is preferably adapted for contacting a fluid scrubbing agent with a vapor phase vent stream from the vent in a counter-current flow configuration and/or adapted to remove heat from the contacting device (2).

In one embodiment, the at least one reactor (1) comprises a first reactor connected to a second reactor for conducting a reactor effluent form the first reactor to the second reactor. The vent of the first reactor may be connected to the contacting device (2) for conducting a vapor phase vent stream from the first reactor to the at least one contacting device (2) and the contacting device (2) may be connected to the second reactor for conducting a stream comprising scrubbing agent combined or reacted with the chlorinating agent and/or a chlorinated compound to the second reactor.

In one embodiment, the first reactor is a continuous stirred tank reactor and in the same, or in an alternative embodiment, the second reactor is a reactor with a plug flow residence time characteristic.

The contacting device (2) may comprise a cooling device adapted for cooling a fluid scrubbing agent and/or cooling a vapor phase vent stream for the vent.

In one embodiment, the vapor-liquid contacting device is connected to an impure chlorinating feed stream for chlorinating a fluid scrubbing agent and the at least one reactor (1) is connected to the at least one contacting device (2) for conducting a chlorinated fluid scrubbing agent effluent from the at least one contacting device (2) to the at least one reactor (1) for introducing chlorinated fluid scrubbing agent effluent into a reaction mixture.

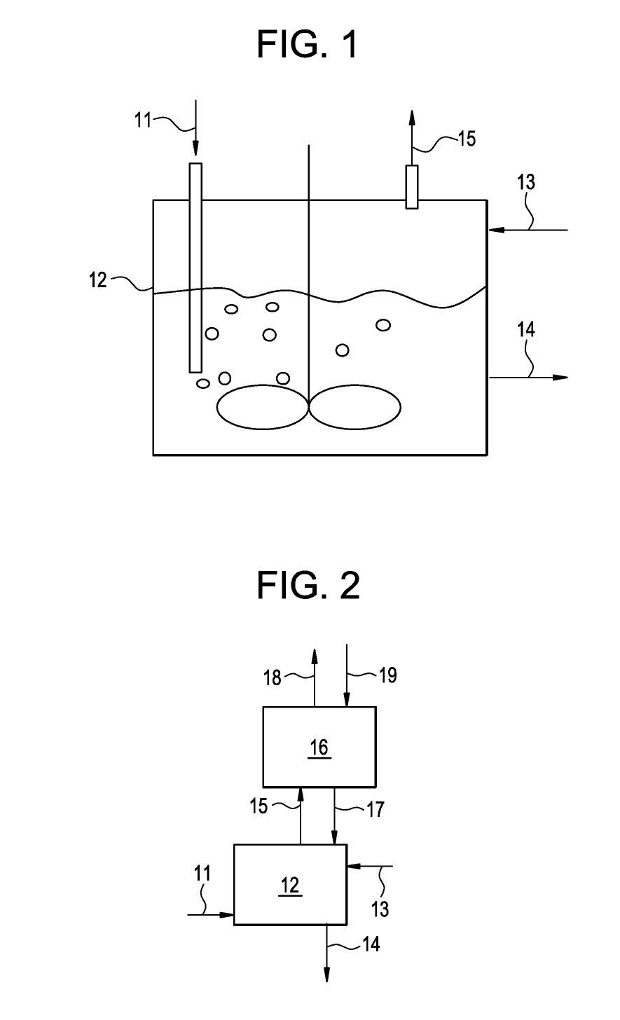

The above process may be conducted using an apparatus according to the present invention. The apparatus is now described in more detail in reference to FIGS. 1 to 5.

FIG. 1 is a schematic diagram showing the main features of an illustrative apparatus that may be used and their respective feed streams.

A hydrochlorination reactor system (12) is fed a gas feed (11) of impure HCl, along with a mixture (13) containing multihydroxylated aliphatic hydrocarbons. The liquid effluent from this reactor system (14) is saturated with HCl at the conditions of the hydrochlorination reactor that it exits. A gas vent (15) leaves reactor (12) with a portion of the HCl feed and any impurities not soluble in the liquid phase and leaving with (14).

FIG. 2 is a block diagram showing the main features of a first embodiment of the present invention.

A hydrochlorination reactor system (12) is fed a gas feed (11) of impure HCl, along with a mixture (13) containing multihydroxylated aliphatic hydrocarbons. Preferably, the HCl gas feed enters the reactor system at a point below the liquid interface, more preferably near the bottom of the reactor, near the level of the agitator or, in the case of a packed or trayed column, near or below the packing or tray support distributor plate. The liquid effluent from this reactor system (14) is saturated with HCl at the conditions of the hydrochlorination reactor that it exits. A gas vent (15) leaves reactor (12) with a portion of the HCl feed and any impurities not soluble in the liquid phase and leaving with (14). In an adsorption unit (16), the vent gas stream (15) is contacted with an adsorptive liquid (19) in such a way that most of the HCl in stream (15) is adsorbed into the adsorptive liquid and is returned to the hydrochlorination reaction system via stream (17). Likewise, the HCl content in stream (18) is substantially reduced from what it was in stream (15).

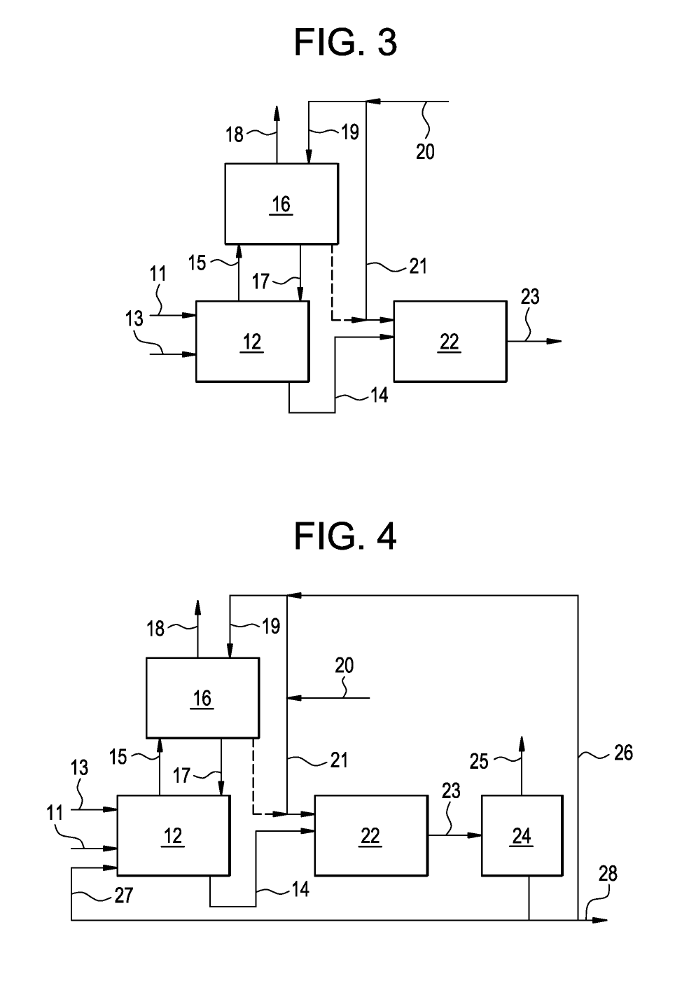

FIG. 3 is a block diagram showing the main features of a second embodiment of an illustrative apparatus that may be used and their respective feed streams.

A hydrochlorination reactor system (12) is fed a gas feed (11) of impure HCl, along with a mixture (13) containing multihydroxylated aliphatic hydrocarbons. Preferably, the HCl gas feed enters the reactor system at a point below the liquid interface, more preferably near the bottom of the reactor, near the level of the agitator or, in the case of a packed or trayed column, near or below the packing or tray support distributor plate. The liquid effluent from this reactor system (14) is saturated with HCl at the conditions of the hydrochlorination reactor that it exits. A gas vent (15) leaves reactor (12) with a portion of the HCl feed and any impurities not soluble in the liquid phase and leaving with (14). In an adsorption unit (16), the vent gas stream (15) is contacted with an adsorptive liquid (19, 20) in such a way that most of the HCl in stream (15) is adsorbed into the adsorptive liquid and is returned to the hydrochlorination reaction system via stream (17). Likewise, the HCl content in stream (18) is substantially reduced from what it was in stream (15). Additionally, if the adsorptive liquid contains multihydroxylated aliphatic hydrocarbons, or other compounds that can react in a beneficial way with HCl, then a portion of the fresh adsorptive liquid (21) may be diverted to a later reactor (22) where the dissolved HCl in stream (14) may react with the MAHCs or other compounds to reduce the amount of HCl in stream (23). Additionally, stream (17) may be optionally diverted to reactor (22) instead of returning to reactor (12).

FIG. 4 is a block diagram showing the main features of a third embodiment of an illustrative apparatus that may be used and their respective feed streams.

A hydrochlorination reactor system (12) is fed a gas feed (11) of impure HCl, along with a mixture (13) containing multihydroxylated aliphatic hydrocarbons. Preferably, the HCl gas feed enters the reactor system at a point below the liquid interface, more preferably near the bottom of the reactor, near the level of the agitator or, in the case of a packed or trayed column, near or below the packing or tray support distributor plate. The liquid effluent from this reactor system (14) is saturated with HCl at the conditions of the hydrochlorination reactor that it exits. A gas vent (15) leaves reactor (12) with a portion of the HCl feed and any impurities not soluble in the liquid phase and leaving with (14). In an adsorption unit (16), the vent gas stream (15) is contacted with an adsorptive liquid (19, 20) in such a way that most of the HCl in stream (15) is adsorbed into the adsorptive liquid and is returned to the hydrochlorination reaction system via stream (17). Likewise, the HCl content in stream (18) is substantially reduced from what it was in stream (15). Additionally, if the adsorptive liquid contains multihydroxylated aliphatic hydrocarbons, or other compounds that can react in a beneficial way with HCl, then a portion of the fresh adsorptive liquid (21) may be diverted to a later reactor(s) (22) where the dissolved HCl in stream (14) may react with the MAHCs or other compounds to reduce the amount of HCl in stream (23). Additionally, stream (17) may be optionally diverted to reactor (22) instead of returning to reactor (12). Stream (23) is separated into a chlorohydrin product containing stream (25) and a recycle stream in separation device(s) (24). That recycle stream may be sent via stream (27) back to the hydrochlorination reactor(s) (12) or via stream (26) to the adsorption unit (16) as the adsorptive fluid for recovering HCl from stream (15). In the case where stream (26) is a primary supply of the adsorptive fluid, then stream (17) is returned to reactor(s) (12) and fresh MAHC in stream 20 may at least partially be diverted to stream (21). Stream (28) is a purge from the recycle to prevent unwanted buildup of undesired heavy components either produced within the hydrochlorination process, or fed as an impurity with the MAHC feed.

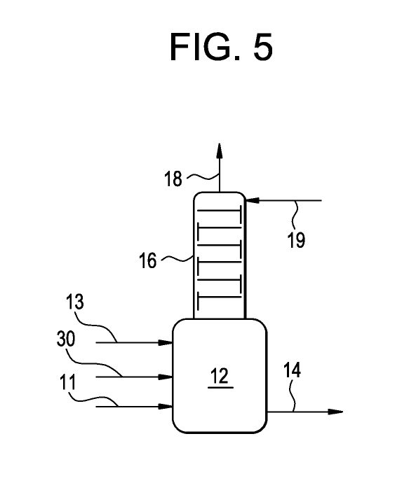

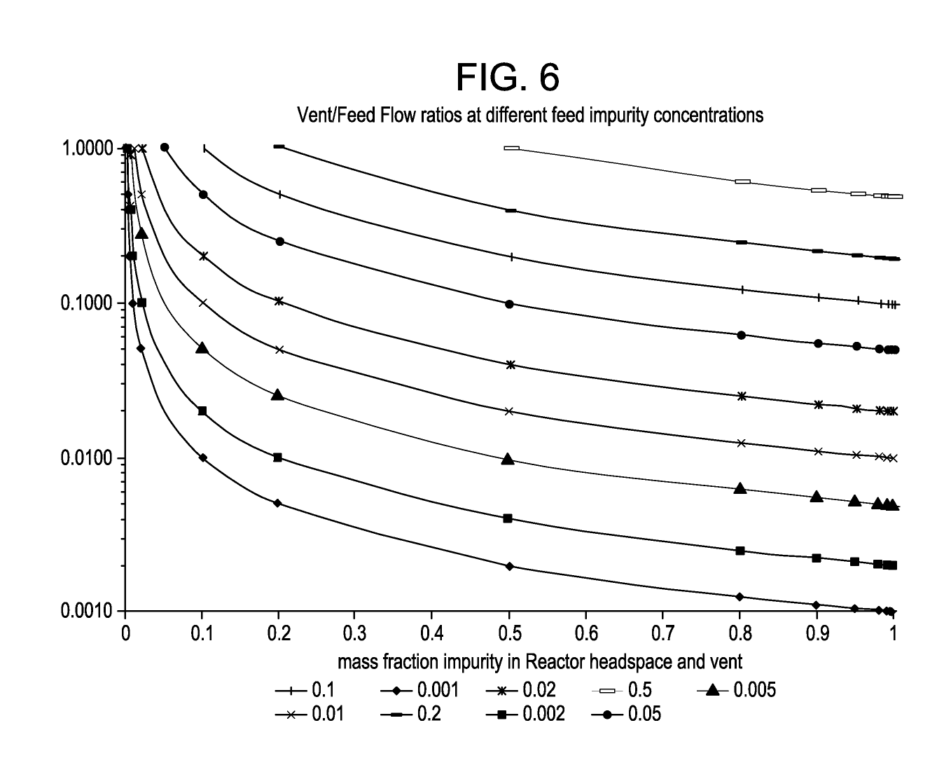

FIG. 5 is a schematic diagram of a specific embodiment of the present invention in which a liquid-vapor contacting device (2) is mounted above the reactor (1).

A hydrochlorination reactor system (12/16) is fed a gas feed (11) of impure HCl, along with a mixture (13) containing multihydroxylated aliphatic hydrocarbons and a catalyst (15). Similarly, the catalyst may be fed as part of stream (13). Preferably, the HCl gas feed enters the reactor system at a point below the liquid interface, more preferably near the bottom of the reactor, near the level of the agitator or, in the case of a packed or trayed column, near or below the packing or tray support distributor plate. The liquid effluent from this reactor system (14) is saturated with HCl at the conditions of the hydrochlorination reactor that it exits. Agitation of the liquid reaction medium in reactor (12) may be accomplished by stirring by an agitator driven by an external motor, or by the injection of the gas stream (11) into the reactor, as with a jet stirred or airlift type reactor. As the gas feed rises through the reactor, it encounters a counter-current stream of reactant containing liquid (in reactor section 16) that contacts the vapor stream and adsorbs reactant HCl up to it's equilibrium content. As it rises, the vapor stream encounters progressively less saturated adsorbent reactive liquid media and at the top of the adsorption section of the reaction the HCl content in stream (18) is substantially reduced from what it was as it rose from reactor section 12 to section 16. The adsorptive liquid (19) may be either a fresh adsorbent liquid, e.g. a solvent, product chlorohydrins, or a fresh MAHC, or a mixture of fresh MAHC and catalyst, or a recycle stream containing MAHC and catalyst along with other compounds.

To the extent that components of the above apparatus are exposed to corrosive materials, such components are preferably fabricated of materials which are resistant to corrosion by the process components. Kirk-Othmer Encyclopedia of Chemical Technology, 2nd Edition (John Wiley and Sons, 1966), volume 11, pages 323-327, presents an extensive discussion of the corrosion resistance of metals and non-metals that can be used in hydrochloric acid and hydrogen chloride service. Specific examples of suitable materials are disclosed in WO 2006/020234. Specific examples include metals such as tantalum, suitable metallic alloys (particularly nickel-molybdenum alloys such as Hastalloy C©), or glass-lined equipment.

The following examples are for illustrative purposes only and are not intended to limit the scope of the present invention.

### Example 1

This example illustrates a process conducted according to the present invention shown in FIG. 2. This example was produced by simulating the present invention using commercially available software and proprietary physical properties, thermodynamics and kinetic models of the major components.

The reactor (12) was simulated as CSTR reactor at 15 bar pressure and 105° C. temperature. The absorber (16) was simulated as falling film absorber operating at 100° C. temperature and 11 bar pressure. The results of the simulation are presented below in Table 1.

TABLE 1

| Stream | 11 | 13 | 14 | 15 | 17 | 18 | 19 |
|---|---|---|---|---|---|---|---|
| Mass Flow kg/hr | 4093 | 17001 | 51339 | 2752 | 18865 | 887 | 17000 |
| Mass Fraction | | | | | | | |
| INERTS | 0.054 | 0.100 | 0.088 | | 0.091 | 0.817 | 0.100 |
| HCl | 0.945 | 0.000 | 0.090 | 0.710 | 0.095 | 0.182 | 0.000 |
| Other Mixed | 0.000 | 0.000 | 0.899 | 0.020 | 0.003 | 0.000 | 0.000 |
| Chlorohydrines | | | | | | | |
| ABSORBENT | 0.000 | 0.900 | 0.010 | 0.000 | 0.811 | 0.000 | 0.900 |

### Example 2

This example illustrates a process conducted according to the present invention as shown in FIG. 3. This example was produced by simulating the present invention using commercially available software and proprietary physical properties, thermodynamics and kinetic models of the major components.

The reactor (12) was simulated as CSTR reactor at 15 bar pressure and 110° C. temperature. The absorber (16) was simulated as absorber operating at 8.5 bar pressure having 3 theoretical stages. The reactor (22) was simulated as an adiabatic plug flow reactor operating at 10 bar pressure. The results of the simulation are presented below in Table 2.

TABLE 2

| | Stream | | | | | | | | | |
|---|---|---|---|---|---|---|---|---|---|---|
| | 11 | 13 | 14 | 15 | 17 | 18 | 19 | 20 | 21 | 23 |
| Total Flow kg/hr | 4094 | 9361 | 51327 | 623 | 10727 | 1115 | 9350 | 17001 | 7650 | 69704 |
| Mass Fraction | | | | | | | | | | |
| INERTS | 0.0550 | 0.0000 | 0.0883 | 0.3625 | 0.0879 | 0.8032 | 0.1000 | 0.1000 | 0.1000 | 0.1099 |
| HCl | 0.9450 | 0.0001 | 0.0899 | 0.6134 | 0.1220 | 0.1968 | 0.0000 | 0.0000 | 0.0000 | 0.0459 |
| Other Mixed | 0.0000 | 0.7819 | 0.9004 | 0.0294 | 0.0934 | 0.0054 | 0.1000 | 0.1000 | 0.1000 | 0.8362 |
| Chlorohydrins | | | | | | | | | | |
| ABSORBENT | 0.0000 | 0.2180 | 0.0095 | 0.0000 | 0.7844 | 0.0000 | 0.9000 | 0.9000 | 0.9000 | 0.1177 |

### Example 3

This example illustrates a process conducted according to the present invention as shown in FIG. 4. This example was produced by simulating the invention using commercially available software and proprietary physical properties, thermodynamics and kinetic models of the major components.

The reactor (12) was simulated as CSTR reactor at 9 bar pressure and 105° C. temperature. The absorber (16) was simulated as absorber operating at 8.5 bar pressure having 3 theoretical stages. The reactor (22) was simulated as an adiabatic plug flow reactor operating at 10 bar pressure. The separation device (24) was simulated as a distillation column operating at pressure 0.07 bar, having 15 theoretical stages with the feed at stage 9. The distillate to reflux ratio was set as 15:1. The boil up ratio in the column was set as 1.55. The results of the simulation are presented below in Table 3.

TABLE 3

| | Stream | | | | |
|---|---|---|---|---|---|
| | 11 | 13 | 14 | 15 | 17 |
| Total Flow kg/hr | 3943 | 11295 | 54998 | 13750 | 45165 |
| Mass Fraction | | | | | |
| INERTS | 0.0550 | 0.0010 | 0.0884 | 0.0884 | 0.0010 |
| HCl | 0.9450 | 0.1081 | 0.0896 | 0.0896 | 0.1082 |
| Other Mixed | 0.0000 | 0.6729 | 0.8977 | 0.8977 | 0.6728 |
| Chlorohydrins | | | | | |
| ABSORBENT | 0.0000 | 0.2188 | 0.0127 | 0.0127 | 0.2189 |
| | Stream | | | | |
| | 18 | 19 | 20 | 21 | 23 |
| Total Flow kg/hr | 874 | 90268 | 4 | 17001 | 71999 |
| Mass Fraction | | | | | |
| INERTS | 0.9887 | 0.0006 | 1.0000 | 0.1000 | 0.1064 |
| HCl | 0.0097 | 0.0603 | 0.0000 | 0.0000 | 0.0397 |
| Other Mixed | 0.0017 | 0.7084 | 0.0360 | 0.1000 | 0.8222 |
| Chlorohydrins | | | | | |
| ABSORBENT | 0.0000 | 0.2312 | 0.0000 | 0.9000 | 0.1380 |

Stream

| | 25 | 26 | 27 | 28 |
|---|---|---|---|---|
| Total Flow kg/hr | 5 | 31706 | 40284 | 201 |
| Mass Fraction | | | | |
| INERTS | 1.0000 | 0.2413 | 0.0000 | 0.0000 |
| HCl | 0.0000 | 0.0899 | 0.0003 | 0.0003 |
| Other Mixed | 0.0000 | 0.9101 | 0.7530 | 0.7530 |
| Chlorohydrins | | | | |
| ABSORBENT | 0.0000 | 0.0000 | 0.2467 | 0.2467 |

### Example 4

This example illustrates a process conducted according to the present invention as shown in FIG. 5. This example was produced by simulating the present invention using commercially available software and proprietary physical properties, thermodynamics and kinetic models of the major components.

The reactor (12) was simulated as CSTR reactor at 11 bar pressure and 110° C. temperature. The absorber (16) was simulated as adiabatic reactive absorber operating 11 bar and having 3 theoretical contact stages with hold up 0.3 m³ each. The results of the simulation are presented below in Table 4.

TABLE 4

| Stream | 13 | 11 | 15 | 14 | 18 | 19 |
|---|---|---|---|---|---|---|
| Mass Flow kg/hr | 9364 | 4093 | 15 | 50502 | 944 | 17001 |
| Mass Fraction | | | | | | |
| INERTS | 0.0000 | 0.0550 | 0.5512 | 0.0860 | 0.9980 | 0.0000 |
| HCl | 0.0000 | 0.9450 | 0.4488 | 0.0840 | 0.0010 | 0.0000 |
| Other Mixed | 0.7820 | 0.0000 | 0.0000 | 0.9040 | 0.0090 | 0.1000 |
| Chlorohydrins | | | | | | |
| ABSORBENT | 0.2180 | 0.0000 | 0.0000 | 0.0120 | 0.0000 | 0.9000 |

### Example 5

Example 5 illustrates performance of an HCl absorber described in the present invention as shown in FIGS. 3, 4 and 5.

A pilot plant CSTR reactor was used to produce a mixed stream of chlorohydrins, water, glycerin and heavier chloroethers. This stream was distilled in a pilot plant distillation column to obtain isomers of dichloropropanol as the top product. The bottom product of the distillation column was used to absorb HCl from a gas stream. The bottom product of the distillation column contained 40% wt isomers of dichloropropanol, 25% wt isomers of chloropropanediol, 21% wt glycerin. The heavier chloroethers and esters of catalyst with the alcohols constituted the rest of the stream. Water constituted <0.1% of the stream.

The bottom product of the distillation column was fed to the top of pilot plant absorber at the rate of 1860 g/hr. The temperature of the stream was 90° C. Gas stream containing 80% wt HCl and 20% wt N2 was fed at the bottom of the column at the rate of 165 g/hr. The temperature of gas stream was 25° C. The pressure at the top of the pilot plant absorber was controlled at 100 psig. The absorber was equipped with a bed of random packing. The height of random packing bed was 1.2 m. In this example, 92% of incoming HCl was absorbed in the pilot plant absorber. The temperature of 92° C. was the highest temperature observed in the packed bed.

### Example 6

Example 6 illustrates performance of an HCl absorber described in the present invention as shown in FIGS. 3, 4 and 5.

A pilot plant CSTR reactor was used to produce a mixed stream of chlorohydrins, water, glycerin and heavier chloroethers. This stream was distilled in a pilot plant distillation column to obtain isomers of dichloropropanol as the top product. The bottom product of the distillation column was used to absorb HCl from a gas stream. The bottom product of the distillation column contained 40% wt isomers of dichloropropanol, 25% wt isomers of chloropropanediol, and 21% wt glycerin. The heavier chloroethers and esters of catalyst with the alcohols constituted the rest of the stream. Water constituted <0.1% of the stream.

The bottom product of the distillation column was fed to the top of pilot plant absorber at the rate of 1899 g/hr. The temperature of the stream was 48° C. Gas stream containing 84% wt HCl and 16% wt N2 was fed at the bottom of the column. The temperature of gas stream was 25° C. The pressure at the top of the pilot plant absorber was controlled at 100 psig. The absorber was equipped with a bed of random packing. The height of random packing bed was 1.2 m. In this example, 80% of incoming HCl was absorbed in the pilot plant absorber. The temperature of 98° C. was the highest temperature observed in the packed bed.

1. A process for making chlorohydrin(s) product(s) comprising contacting a liquid-phase reaction mixture comprising one or more of multihydroxylated-aliphatic hydrocarbon compound(s) and/or ester(s) thereof and/or monochlorohydrin(s) and/or ester(s) thereof with at least one chlorinating feed stream comprising at least one chlorinating agent and at least one impurity having a boiling point below the boiling point of the chlorohydrin product having the lowest boiling under hydrochlorination conditions, optionally in the presence of water, one or more catalyst(s), and/or one or more heavy byproduct(s) in at least one reaction vessel under hydrochlorination conditions; wherein (a) the liquid-phase reaction mixture is maintained at a temperature below the boiling point of the chlorohydrin product having the lowest boiling point under hydrochlorination conditions and greater than the boiling point(s) of the at least one impurity; and (b) a vapor phase vent stream comprising the at least one impurity having a boiling point below the boiling point of the chlorohydrin product having the lowest boiling under hydrochlorination conditions is removed from the liquid-phase reaction mixture.

2. The process according to claim 1, wherein (i) the multi-hydroxylated aliphatic hydrocarbon compound(s) comprises glycerin and/or monochlorohydrin(s); (ii) the monochlorohydrin(s) comprises 3-chloro-1,2-propanediol and/or 2-chloro-1,3-propanediol; and (iii) the dichlorohydrin(s) comprise(s) 1,3-dichloro-2-propanol and/or 2,3-dichloro-1-propanol.

3. The process according to claim 1, wherein the at least one chlorinating feed stream comprises (i) a byproduct of the manufacture of substances other than chlorohydrin(s); (ii) a byproduct of a chemical reaction in which phosgene is one of the reactants; (iii) a byproduct of the manufacture of toluene diisocyanate, methylene-di(phenylisocyanate) and/or polymeric methylene di(phenylisocyanate); (iv) a byproduct of a chemical reaction or series of chemical reactions in which ethylene or propylene is one of the reactants; (v) a byproduct of the simultaneous manufacture of 1,3-dichloropropene and allyl chloride; (vi) a byproduct of a chemical reaction in which ethylene chloride is one of the reactants; (vii) a byproduct of the manufacture of vinyl chloride; (viii) a reaction product of a chlorinated hydrocarbon and hydrogen; or (ix) wherein the chlorinating agent comprises a source of hydrogen chloride.

4. The process according to claim 1, wherein the at least one impurity comprises one or more vapors that are nonreactive with the reaction mixture under hydrochlorination conditions; or wherein the at least one chlorinating feed stream contains impurities that are not reactive to the chlorohydrins product or the reactants used to produce chlorohydrins product under hydrochlorinating conditions; and wherein the at least one impurity comprises nitrogen, carbon monoxide, carbon dioxide, phosgene, chlorinated organic compound(s), hydrogen, chlorine, water, amine, ammonia, methanol, aliphatic hydrocarbon compound(s), or olefinic hydrocarbon compound(s), or a combination thereof.

5. (canceled)

6. (canceled)

7. The process according to claim 4 wherein a reaction step occurs by contacting a multihydroxylated-aliphatic hydrocarbon, an ester of a multihydroxylated-aliphatic hydrocarbon, or a mixture thereof with a source of a superatmospheric partial pressure of hydrogen chloride, in the presence of a catalyst to produce a chlorohydrin, an ester of a chlorohydrin, or a mixture thereof, said contacting step carried out without substantial removal of water; and wherein the at least one chlorinating feed stream entering the reaction step is at superatmospheric partial pressure of hydrogen chloride.

8. (canceled)

9. (canceled)

10. The process according to claim 1, wherein the at least one chlorinating feed stream is introduced into the reaction mixture in the vapor phase; and wherein the partial pressure of the chlorinating agent introduced into the reaction vessel is greater than the partial pressure of the chlorinating agent in the vapor phase vent stream.

11. (canceled)

12. The process according to claim 1, wherein a feed flow rate of the chlorinating agent introduced into the at least one reaction vessel is more than 1 percent greater than a flow rate of the chlorinating agent in the vapor phase vent stream.

13. The process according to claim 1, wherein the at least one reaction vessel is a continuous stirred tank reactor, a tubular reactor, a cylindrical reactor, a bubble column reactor, a packed tower, a trayed tower, a spray tower, a venturi eductor, a heat exchanger, a falling film contactor or any combination thereof.

14. The process according to claim 1, including:
(c) the vapor phase vent stream is contacted, in a liquid-vapor contacting device, with a fluid scrubbing agent capable of absorbing or reacting with the chlorinating agent(s) present in the chlorinating feed stream to remove chlorinating agent(s) from the vapor phase vent stream; or
(c′) the chlorinating feed stream is contacted, in a liquid-vapor contacting device, with a fluid scrubbing agent capable of absorbing or reacting with the chlorinating agent(s) present in the chlorinating feed stream to remove chlorinating agent(s) from the chlorinating feed stream.

15. The process of claim 14, wherein the vapor phase vent stream entering the absorption step (c) is at superatmospheric partial pressure of hydrogen chloride; and wherein the fluid scrubbing agent of step (c) comprises a solvent; and wherein the solvent comprises at least one of an alcohol, a halogenated fluid, an ether, a saturated hydrocarbon, an aromatic hydrocarbon, or mixtures thereof.

16. (canceled)

17. The process according to claim 14, wherein the fluid scrubbing agent is capable of reacting with the chlorinating agent from the vapor phase vent stream to form chlorohydrin(s); and wherein the fluid scrubbing agent comprises (i) at least one multi-hydroxylated aliphatic hydrocarbon compound and/or ester thereof; (ii) at least one monochlorohydrin or ester thereof; (iii) at least one multi-hydroxylated aliphatic hydrocarbon and/or monochlorohydrin, and/or ester(s) thereof; or (iv) at least one catalyst or ester thereof, contained in the reaction mixture.

18. (canceled)

19. The process according to claim 14, wherein the reaction mixture is removed from the reaction vessel; at least a portion of the chlorinating agent(s) and/or a portion of dichlorohydrin(s) present in the reaction mixture are removed from the reaction mixture in one or more unit operations; and the reaction mixture residue depleted of chlorinating agent(s) and a portion of dichlorohydrin(s) is used as the fluid scrubbing agent.

20. The process according to claim 14, wherein the fluid scrubbing agent is introduced as a feed stream flowing in counter-current flow direction relative to the vapor phase vent stream or chlorinating feed stream; or wherein the fluid scrubbing agent is introduced into at least one downstream reaction vessel connected to the at least one reaction vessel that is the source of the vapor phase vent stream; and wherein the at least one downstream reaction vessel comprises at least one reactor having a plug flow residence time characteristic.

21. The process according to claim 14, wherein the contacting is carried out via reactive distillation.

22. The process according to claim 14, including:
(d) at least a fraction of the fluid scrubbing agent containing the absorbed or reacted chlorinating agent(s) is introduced into the at least one reaction vessel after step (c) or (c′).

23. The process according to claim 22, wherein the reaction vessel is the source of the vapor phase vent stream and at least a fraction of the fluid scrubbing agent containing the absorbed or reacted chlorinating agent(s) from the liquid-vapor contacting device is introduced into the at least one reaction vessel that is the source of the vapor phase vent stream for the liquid-vapor contacting device according to step (c); and wherein the source of the vapor phase vent stream is (i) a continuous stirred tank reactor; (ii) a bubble column reactor; (iii) a trayed distillation tower reactor; or (iv) a flash vessel downstream from the at least one reactor.

24. (canceled)

25. (canceled)

26. (canceled)

27. The process according to claim 1, wherein the steps of the process are carried out simultaneously and continuously for a predetermined period of time.

28. An apparatus for making chlorohydrin(s) comprising:
(1) at least one reactor suitable for introducing a chlorinating feed stream comprising at least one chlorinating agent and at least one impurity having a boiling point below the boiling point of the chlorohydrin product having the lowest boiling under hydrochlorination conditions, into a liquid reaction mixture comprising at least multihydroxylated-aliphatic hydrocarbon compound(s) and/or ester(s) thereof and/or chlorohydrin(s) and/or ester(s) thereof; and
(2) at least one vapor-liquid contacting device, connected to the at least one reactor (1), for recovering chlorinating agent from the reaction mixture present in the at least one reactor (1); said chlorinating agent recovered for recycle to the at least one reactor (1);
wherein the at least one reactor (1) has at least one vent for removing a vapor phase vent stream from within the reactor (1), said vapor vent stream comprising at least one chlorinating agent and at least one impurity having a boiling point below the boiling point of the chlorohydrin product having the lowest boiling under hydrochlorination conditions;
the at least one vapor-liquid contacting device (2) is connected to the at least one reactor (1) for conducting a stream comprising a fluid scrubbing agent combined and/or reacted with chlorinating agent to the at least one reactor (1); and
the at least one vent is connected to the at least one vapor-liquid contacting device (2) for conducting the vapor phase vent stream from the at least one reactor (1) to the at least one vapor-liquid contacting device (2) for contacting the vapor phase vent stream with the fluid scrubbing agent for removing chlorinating agent from the vapor phase vent stream; and/or
the at least one reactor (1) is connected to the at least one vapor-liquid contacting device (2) for conducting a chlorinated fluid scrubbing agent effluent from the at least one vapor-liquid contacting device (2) to the at least one reactor (1) for introducing the chlorinated fluid scrubbing agent effluent into the reaction mixture present in the at least one reactor (1).

29. The apparatus according to claim 28, further comprising at least one separation vessel (3) for separating at least a portion of the chlorinating agent(s) and/or a portion of the dichlorohydrin(s) from unreacted and partially reacted reactants; said separation vessel (3) connected to the at least one reactor (1) for conducting a liquid reaction mixture effluent stream from the at least one reactor (1) to the at least one separation vessel (3).

30. The apparatus according to claim 28, wherein the at least one separation vessel (3) is connected to the at least one vapor-liquid contacting device (2) for conducting a stream comprising at least a fraction of a liquid residue after separation of chlorinating agent(s) and dichlorohydrin(s), from the at least one separation vessel (3) to the at least one vapor-liquid contacting device (2) for use as a fluid scrubbing agent for removing chlorinating agent from a vapor phase vent stream.

31. The apparatus according to claim 28, wherein the at least one vapor-liquid contacting device (2) comprises a falling film absorber, a heat exchanger, a scrubbing column; or any combination thereof.

32. The apparatus according to claim 28, wherein the at least one vapor-liquid contacting device (2) is adapted for contacting a fluid scrubbing agent with a vapor phase vent stream from the vent in a counter-current flow configuration; or wherein the at least one vapor-liquid contacting device (2) is adapted to remove heat from the at least one vapor-liquid contacting device (2).

33. (canceled)

34. The apparatus according to claim 28, wherein the at least one reactor (1) comprises (i) a continuous stirred tank reactor; (ii) a bubble column reactor; or (iii) a trayed distillation tower reactor; or wherein the at least one reactor (1) comprises a first reactor connected to a second reactor for conducting a reactor effluent from the first reactor to the second reactor.

35. (canceled)

36. The apparatus according to claim 28, wherein the vent of the first reactor is connected to the at least one vapor-liquid contacting device (2) for conducting a vapor phase vent stream from the first reactor to the at least one contacting device (2); and the at least one vapor-liquid contacting device (2) is connected to the second reactor for conducting a stream comprising scrubbing agent combined or reacted with the chlorinating agent and/or a chlorinated compound to the second reactor; and wherein the first reactor is a continuous stirred tank reactor; and the second reactor is a reactor with a plug flow residence time characteristic.

37. (canceled)

38. The apparatus according to claim 28, wherein the at least one vapor-liquid contacting device (2) comprises a cooling device adapted for cooling a fluid scrubbing agent and/or cooling a vapor phase vent stream from the vent; or wherein the at least one vapor-liquid contacting device (2) is connected to an impure chlorinating feed stream for chlorinating a fluid scrubbing agent; and the at least one reactor (1) is connected to the at least one vapor-liquid contacting device (2) for conducting a chlorinated fluid scrubbing agent effluent from the at least one vapor-liquid contacting device (2) to the at least one reactor (1) for introducing chlorinated fluid scrubbing agent effluent into the reaction mixture.

39. (canceled)
